(19) United States
(12) Patent Application Publication
(10) Pub. No.: US 2003/0199457 A1
(43) Pub. Date: Oct. 23, 2003

(54) Prevention and treatment of thromboembolic disorders associated with arterial & venous thrombosis

(76) Inventors: Mawaheb M. El-Naggar, Lincoln University, PA (US); Shaker A. Mousa, Lincoln University, PA (US)

Correspondence Address: Dr. Mawaheb M. El-Naggar, 7 Linden Circle, Lincoln University, PA, 19352, US

(21) Appl. No.: 10/123,959

(22) Filed: Apr. 17, 2002

(51) Int. Cl. A61K 31/70

(52) U.S. Cl. 514/23

(57) ABSTRACT

This invention provides for the treatment of thrombosis through the administration, in combination, of a short or long acting GPIIb/IIIa antagonist (A) and anticoagulant (B) that might include the following: UFH or low molecular weight heparin (LMWH), ultra LMWH, pentasaccharide or direct anti-Xa or direct anti-IX/IXa, direct anti-IIa (thrombin) or tissue factor pathway inhibitor (TFPI) for the prevention and treatment of thromboembolic events associated with arterial or venous thrombosis and other thrombosis-induced processes by any means that produces contact of the agents with their site of action wherein at least one of the components of each of these combinations is given in reduced amount.

### FIELD OF THE INVENTION

This invention relates to the prevention and treatment of thromboembolic disorders in mammals, and more particularly to such treatment by the administration of a lyophilized formulation of a platelet GPIIb/IIIa antagonist, and anticoagulant such as unfractinoated heparin, low molecular weight heparin (LMWH), ultra LMWH, pentasaccharide, direct anti-Xa, direct anti-IIa (thrombin), anti-VIIa, anti-tissue factor or recombinant tissue factor pathway inhibitor (r-TFPI) at sub-therapeutic levels.

### BACKGROUND OF THE INVENTION

Intravascular thrombosis is one of the most frequent pathological events and a major cause of morbidity and mortality in western civilization. Factors that stimulate thrombosis include vascular damage, stimulation of platelets, and activation of the coagulation cascade. Platelet adhesion to exposed sub-endothelial surfaces of injured vessels with subsequent activation and the resulting aggregation and activation of coagulation system has been shown to be associated with various vascular pathological conditions (Mousa, Drug discovery today 4: 552-561, 1999; Mousa and Bennett, Drugs of the Future 21: 1141-1154, 1996; Bennett and Mousa, Thrombosis & Haemostasis, 85:1-6, 2001).

Antiplatelet therapy has become a standard treatment of acute and chronic arterial thrombotic diseases. Among current available anti-platelet drugs, aspirin is the drug of choice for secondary prevention of myocardial infarction (Schror, Drugs 50:7-28, 1995). Its antiplatelet activity is mainly due to the irreversible inhibition of the platelet cyclo-oxygenase causing a last-lasting blockade of platelet-dependent thromboxane A₂ formation. Since in vivo platelet aggregation is stimulated by several agonists including thromboxane A₂, ADP and thrombin, blockade of the formation of thromboxane A₂ by aspirin may not be sufficient for a complete inhibition of platelet aggregation.

The final common step in platelet aggregation, regardless of the stimulus, involves the interaction of adhesive proteins such as fibrinogen and vWf with the platelet membrane GPIIb/IIIa (Pytela et al., Science 231:1559-1562, 1986; Philips et al., Cell 65:359-362, 1991). It is now well established that the binding of fibrinogen to the GPIIb/IIIa receptor on activated platelets is considered as the final common pathway of platelet aggregation (Mousa, Drug discovery today 4: 552-561, 1999; Mousa and Bennett, Drugs of the Future 21: 1141-1154, 1996; Bennett and Mousa, Thrombosis & Haemostasis, 85:1-6, 2001).

Thus, blockade of fibrinogen binding to the GPIIb/IIIa receptor on activated platelet should inhibit platelet aggregation induced by all agonists. Peptide, peptidomimetic and non-peptide GPIIb/IIIa antagonists have been developed, and their anti-thrombotic effects have been well demonstrated (Mousa, Drug discovery today 4: 552-561, 1999; Mousa and Bennett, Drugs of the Future 21: 1141-1154, 1996). All active small molecule GPIIb/IIIa antagonists either peptide, peptidomimetics or non-peptide are zwitterionic, with carboxyl ate based negative charge and cationic amine based positive charge.

Several studies have identified the pivotal role of GPIIb/IIIa receptor in coronary thrombosis, and this platelet integrin receptor has emerged as a rational therapeutic target in the management of acute coronary syndromes (The EPIC Investigators, N Engl J Med., 330: 956-961, 1994; Mousa and Topol, Current review of Interventional Cardiology, 3^{rd} edition, Current Medicine, 13: 114-129, 1997). Studies in man with a monoclonal antibody for GPIIb/IIIa (c7E3, Abciximab) have suggested the antithrombotic benefit of GPIIb/IIIa antagonism, which is in agreement with the initial preclinical investigation in animal model (The EPIC Investigators, N Engl J Med., 330: 956-961, 1994). Intravenous administration of c7E3 Fab antibody (abciximab, ReoPro) in high-risk patients undergoing angioplasty has been shown to reduce the composite incidence of major ischemic events (Coller et al., Blood 68: 783-786, 1986). In other clinical studies, Abciximab demonstrated efficacy when given in combination with thrombolytic therapy and in refractory unstable angina patients prior to angioplasty (Tcheng, Thrombosis & Haemostasis, 78:205-209, 1997. Additionally, several other selective GPIIb/IIIa antagonists, including Integrilin and Tirofiban (MK383) are small molecule GPIIb/IIIa antagonists for intravenous use in the treatment and prevention of acute ischemic heart diseases in the settings of angioplasty, thrombolysis and unstable angina (Peerlinck et al., Circulation 88: 1512-1517, 1993; Tcheng et al., Circulation 91: 2151-2157, 1995). Current intravenous GPIIb/IIIa antagonists in clinical use such as Tirofiban or Integrilin have a faster rate of dissociation from human platelets reflecting their short duration of antiplatelet effects as compared to that of Abciximab (Mousa and Bennett, Drugs of the Future 21: 1141-1154, 1996).

Clinical studies with orally active GPIIb/IIIa antagonists including Xemilofiban, Orbofiban, Sibrafiban, Lotrafiban, and LeFradafiban demonstrated variable oral antiplatelet activity in man upon their administration (Mousa, Drug discovery today 4: 552-561, 1999; Simpfendorfer et al., Circulation 96: 76-81, 1997). In contrast to the success of IV GPIIb/IIIa antagonists, recent clinical trials demonstrated lack of clinical benefit for the oral delivery of GPIIb/IIIa antagonists. Additionally, a second generation oral GPIIb/IIIa antagonists with tight binding to GPIIb/IIIa receptors along with slow dissociation rate such as Roxifiban (Mousa and Bennett, Drugs of the Future 21: 1141-1154, 1996; Mousa et al., Coronary Artery Disease 7: 767-774, 1996; Mousa et al., J Pharmacol Exp Thera., 286:1277-1284, 1998) might provide improved pharmacodynamic were discontinued in light of the failure of the first generation oral agents (Simpfendorfer et al., Circulation 96: 76-81, 1997; Muller et al., Circulation 96: 1130-1138, 1997; Cannon et al., Circulation 97: 340-349, 1998; Cannon et al., Circulation 102: 149-156, 2000). The success of IV GPIIb/IIIa antagonists might be dependent on the use of an anticoagulant such as heparin, which was not included in the oral formulation.

Anticoagulants:

Heparin and LMWH: Heparin and LMWH are polyanionic glycosaminoglycan, with carboxylate and sulfate functions. Compared to unfractionated heparin (UFH), LMWHs exhibit improved subcutaneous (SC) bioavailability; lower protein binding; longer half-life; variable number of antithrombin III binding sites; variable glycosaminoglycan contents; variable anti-serine protease activities (anti-Xa, anti-IIa); variable potency in releasing TPFPI (Young et al., Thromb Haemost 71: 300-304, 1994; Frydman, Haemost 26: 24-38, 1996; Fareed et al., Am J Cardiol 82: 3L-10L, 1988). For these reasons, over the last decade LMWHs have increasingly replaced UFH in the prevention and treatment of venous thromboembolic disorders (VTE) because of its pharmacoeconomic advantages over UFH (Hull et al., Thromb Haemost 24: 21-31, 1998; Hull et al., N Engl. J Med. 329: 1370-1376, 1993; Levine et al., N Engl. J Med. 334: 677-681, 1996; Hirsh, Semin Hematol., 34: 20-25, 1999; Simonneau et al., N Engl. J Med. 337: 663-669, 1997). Randomized clinical trials have demonstrated that individual LMWHs used at optimized dosages are at least as effective as and probably safer than UFH. The convenient once- or twice daily SC dosing regimen without the need for monitoring has encouraged the wide use of LMWHs. It is well established that different LMWHs vary in their physical and chemical properties due to the differences in their methods of manufacturing. These differences translate into differences in their pharmacodynamic and pharmacokinetic characteristics (Fareed et al., Ann N.Y. Acad Sci. 556: 333-353, 1989). The World Health Organization (WHO) and United States Food and drug administration (US-FDA) regard LMWHs as individual drugs that cannot be used interchangeably (Fareed et al., Ann N.Y. Acad Sci. 556: 333-353, 1989; Simonneau et al., N Engl. J Med. 337: 663-669, 1997).

Other Anticoagulants:

Recently a synthetic pentasaccharide (indirect anti-Xa) was developed for the prevention and treatment of venous thromboembolic disorders and certain settings of arterial thrombosis (Hirsh and Weitz, Lancet, 93:203-241, 1999). Additionally, various direct anti-Xa were synthesized and advanced to clinical development (Phase I-II) for the prevention and treatment of venous thromboembolic disorders and certain settings of arterial thrombosis (Hirsh and Weitz, Lancet, 93:203-241, 1999; Nagahara et al., Drugs of the Future, 20: 564-566, 1995; Pinto et al., 44: 566-578, 2001; Pruitt et al., Biorg Med Chem Lett, 10: 685-689, 2000; Quan et al., J Med Chem 42: 2752-2759, 1999; Sato et al., Eur J Pharmacol, 347: 231-236, 1998; Wong et al, J Pharmacol Exp Thera, 292: 351-357, 2000). A direct anti-IIa (thrombin) such as Xemilegtran is in Phase II-III of clinical development in venous and certain settings of arterial thrombosis (Hirsh and Weitz, Lancet, 93:203-241, 1999; Fareed et al., Current Opinion in Cardiovascular, pulmonary and renal investigational drugs, 1:40-55, 1999). Additionally, a number of anti-VIIa and anti-tissue factor are in pre-clinical and early stage of clinical development. Furthermore recombinant tissue factor pathway inhibitor (r-TFPI) is under pre-clinical and clinical investigations for a number of years (Kaiser et al, Emerging Drugs 5:73-87, 2000; G Hirsh and Weitz, Lancet, 93:203-241, 1999; Bajaj and Bajaj, Thromb Haemost, 78: 471-477, 1997; Roque et al, J Am Coll Cardiol, 36: 2303-2310, 2000).

Platelet, Coagulation & Inflammatory Stimuli: Several lines of evidences demonstrated the interplay between the platelet in the activated state and the coagulation cascade. Platelet upon activation, due to injured blood vessels, mechanically or pathophysiologically, with the exposure of sub-endothelium, exposure of collagen and VFW leading to platelet adhesion and its activation. That led to the exposure of the platelet GPIIb/IIIa receptors in its active state leading to platelet fibrinogen binding and amplification of platelet aggregate formation. Additionally, activated platelets interact with leukocyte leading to platelet-leukocyte cohesion and leukocyte activation. Hyperactive platelets also provide a surface for thrombin generation, a potent platelet activator. Additionally, there is a significant interplay between the coagulation cascade, platelet and the vessel wall in the promotion of thromboembolic disorders. Depending upon the shear level venous (low shear) versus arterial (high shear), platelet/fibrin proportions and contributions vary.

For example, infection leading to the initiation of pro-inflammatory stimuli could be a major predisposing factor in propagation of thromboembolic disorders. Endotoxin that can be liberated from E. coli and other bacteria, can induce pro-inflammatory state with the increase of TNF alpha and other cytokines. That would lead to the activation of leukocyte with the increased expression of membrane L-selectin, and the shedding of soluble L-selectin, which can serve as a surrogate marker of leukocyte activation. Activation of leukocyte leads to the propagation and generation of tissue factor, which initiate and amplify a hyper-coaguable state. A hyper-coaguable state with the generation of thrombin, activate the platelets leading to the over-expression of platelet membrane p-selectin and the shedding of soluble p-selectin which can act as a potential surrogate marker of platelet activation. Additionally, the pro-inflammatory state can induce endothelial cell (EC) insult leading to increased EC membrane expression and shedding of soluble vascular adhesion molecules-1 (VCAM-1), intracellular adhesion molecule-1 (ICAM-1) and E-selectin.

### METHODS

Different techniques were used. These include the following: Thrombelastography for platelet-fibrin clot dynamic studies and Light transmittance aggregometry for platelet-platelet interaction studies.

Thrombelastography: Clot formation was monitored at 37° C. in an oscillating plastic cylindrical cuvette (“cup”) and a coaxially suspended stationary piston (“pin”) with a 1 mm clearance between the surfaces, using a computerized Thrombelastograph (CTEG Model 3000, Haemoscope, Skokie, Ill.) as described by Mousa et al., Athero Thromb Vasc Biol 2000. The cup oscillates 4°45′ (1/12 radian) in either direction every 4.5 seconds, with a 1 second mid-cycle stationary period; resulting in a frequency of 0.1 Hz and a maximal shear rate of 0.1 per second. The pin is suspended by a torsion wire that acts as a torque transducer. With clot formation, fibrin fibrils physically link the cup to the pin and the rotation of the cup (Transmitted to the pin) is displayed on-line using an IBM-compatible personal computer and customized software (Haemoscope Corp., Skokie, Ill.). The torque experienced by the pin (relative to the cup's oscillation) is plotted as a function of time (Essel et al., J Cardiovascular Anesth., 7:410-415, 1993; Mousa et al., Arteriosclerosis Thromb Vasc Biol., 20:1162-1167, 2000). The amplitude on the TEG tracing is a measure of the rigidity of the clot; the peak strength or the shear elastic modulus attained by the clot, G, is a function of clot rigidity and can be calculated from the maximal amplitude (MA) of the TEG (Mousa et al., Arteriosclerosis Thromb Vasc Biol., 20:1162-1167, 2000).

Platelet Contribution to Clot Strength: To assess the effect of the TF modification on peak clot strength (G) was measured with and without the addition of TF in paired samples of WB and PRP. To document the contribution of activated platelets to the elastic modulus of fibrin clots, the effect of platelet number was measured by serially diluting PRP with PPP. The platelet count of each dilution was measured before performing TEG. Tissue factor-triggered TEG was performed with increasing concentrations added to the TEG cups. The effect of GPIIb/IIIa blockade on clot strength was studied by adding increasing concentrations of c7E3 Fab (Abciximab), cyclic peptide, peptidomimetic and non-peptide GPIIb/IIIa antagonists (Mousa and Bennett, Drugs of the Future 21: 1141-1154, 1996; Mousa et al., Athero Thromb Vasc Biol., 20:1162-1167, 2000) and various LMWH to the TEG cup along with CaCl₂ and TF. The Thrombelastograph maximum amplitude (MA) for platelets MA (PLT)) was calculated by subtracting the MA from a platelet-poor plasma (PPP) sample MA (PPP) determined in one TEG well from that of whole blood MA (WB) run simultaneously in the second TEG well.

The most potent GPIIb/IIIa antagonists, Abciximab, XV454, DMP802, XV459 and its ester pro-drug roxifiban resulted in 80% maximal blockade, which mimic platelet removal from whole blood. In contrast, LMWH such as tinzaparin, enoxaparin, fraxiparin, dalteparin or direct thrombin inhibitors, direct anti-Xa or TFPI and other anticoagulants resulted in a dose response inhibitory efficacy, with 100% blockade of TF-induced clot formation/strength (Mousa, Thromb Haemost 2000).

UFH and LMWHs act at multiple sites; Heparin has a plasmatic as well as vascular affect. LMWH act at different levels in inhibiting factor Xa activity, thrombin (IIa) activity, and in releasing tissue factor pathway inhibitor (TFPI). The above listed techniques were used in order to investigate the interaction between LMWH and GPIIb/IIIa receptor antagonists.

Platelet Aggregation (Light Transmittance Aggregometry): Agonist-induced platelet aggregation was measured as change in % light transmission of PRP (platelet count 2×10⁵ per μL). For studying the effect of LMWH & GPIIb/IIIa antagonists on platelet aggregation, increasing concentrations were added to PRP for 5 minutes after which 0.5 IU/ml thrombin was added. The aggregation response was measured as the maximum response of the increase in light transmission induced by thrombin, using PPP to establish 100% light transmission (Mousa et al., Coronary Artery Disease 7: 767-774, 1996). IC50s value for the different LMWH and GPIIb/IIIa antagonists were determined. The effects of sub-therapeutic concentrations of the platelet GPIIb/IIIa antagonists, Roxifiban or Abciximab on the dose-response relationship of LMWH and vice versa demonstrated a significant enhancement (synergy) of anti-thrombotic efficacy (Mousa., Thrombosis & Haemostasis 1999).

For the platelet GPIIb/IIIa antagonist, Abciximab the pivotal EPIC and EPILOG trials documented the potential clinical benefit of Abciximab in ACS (The EPIC Investigators, N Engl. J Med. 330: 956-961, 1994). In the EPIC trial, there was a significant excessive bleeding where unfractionated heparin was used in its full dose with Abciximab. That led to the EPILOG trial where a reduced dose of heparin was used leading to improved safety profile and without compromising the achieved efficacy in EPIC trial (The EPIC Investigators, N Engl. J Med. 330: 956-961, 1994; Epilog Investigators., N Engl J Med., 336: 1689-1696, 1997). From the efficacy aspect, the PRISM and PRISM PLUS trial with Tirofiban plus heparin versus Tirofiban arm; the Tirofiban plus heparin arm was more significantly effective than Tirofiban alone, again suggesting an improved efficacy when you have heparin on board with GPIIb/IIIa antagonist (RISM study Investigators., N Engl J Med 338:1498-1505, 1998; PRISM-PLUS study Investigators., N Engl J Med., 338: 1488-1497, 1998). These are two key clinical evidences, and many more are actually under investigation demonstrating a strong rational for the combination of GPIIb/IIIa antagonist with adjusted doses of heparin. We know the advantage of LMWH over UFH, so it seems that LMWH with platelet GPIIb/IIIa receptor antagonists might have a potential benefit when combined at the right dose regimens.

In all of IV GPIIb/IIIa antagonist trials, the GPIIb/IIIa antagonists and UFH were given by IV bolus followed by IV infusion as a separate product)

The co-adminstration of intravenous GPIIb/IIIa antagonist (BSPBPA) at sub-effective or reduced levels with the LMWH enoxaparin resulted in enhanced anti-thrombotic effect. This was not the case with standard heparin when co-administered at the same reduced level of the GPIIb/IIIa antagonists BSPBPA (U.S. Pat. No. 6,103,705; 2000). The conclusion for the differences were explained based onthe anti-Xa activity of enoxaparin. In another case, it was shown that co-administration of LMWH such as tinzaparin with certain GPIIb/IIIa antagonists resulted in enhanced efficacy (U.S. Pat. No. 2,006,8719 A1; 2002).

Low molecular weight heparins are obtained from standard, unfractionated heparin, are as effective as standard, unfractionated heparin for prophylaxis and treatment of venous thromboembolism and have fewer side effects. The current available low molecular weight heparins include, for example, tinzaparin, certoparin, parnaparin, nadroparin, ardeparin, enoxaparin, reviparin, dalteparin and fraxiparin.

Safety Advantages for the Combination of GPIIb/IIIa Antagonists and Heparin:

It has been demonstrated that the platelet GPIIb/IIIa antagonist can effectively inhibit Heparin-induced platelet activation in plasma from heparin-induced thrombocytopenia (HIT) patients and in patients with HIT (Walenga et al., Clin Appl Thromb Haemost 3: S53-63, 1997; Jeske et al., Thromb Res., 88:271-281, 1997; Walenga et al., Hamostaseologie, 19: 128-133, 1999; Mousa et al., J Am Coll Cardiol, 35: 1178, 317A, 2000), which would result in serious and fatal thrombotic thrombocytopenia. Furthermore, plasma from patients who developed thrombocytopenia after GPIIb/IIIa antagonist that result in increased platelet activation and secretion could be blocked by anticoagulants such as direct or indirect thrombin inhibitors. Hence the combination of both the platelet GPIIb/IIIa antagonist and anticoagulant such as UFH, LMWH, anti-Xa, anti-IX/IXa, anti-IIa, TFPI, ultra-LMWH, pentasaccharide, and other anticoagulants would result in a mutually safer formulation with less thrombocytopenia that could either the result of heparin, LMWH or the GPIIb/IIIa antagonist.

Formulation of zwitterionic GPIIb/IIIa antagonists with polyanionic heparin requires the addition of polycationic carbohydrate such as Chitosan or polycationic peptides in the presence of citric acid, sodium citrate, mannitol, and other non-active ingredients.

Formulation of zwitterionic GPIIb/IIIa antagonists with small molecules anti-Xa, anti-IIa, and other anticoagulants requires the addition of sodium caproate in the presence of citric acid, sodium citrate, mannitol, and other non-active ingredients.

### SUMMARY OF THE INVENTION

One object of the present invention is to provide a method of treating thrombosis and cancer in a mammal comprising: administering the combination in a therapeutically effective amount of (i) a GPIIb/IIIa antagonist selected from the group consisting of Abciximab, XV454, XV459, DMP802, roxifiban (class I) as defined by Mousa et al., Athero Thromb Vasc Biol., 2000) and eptifibatide, tirofiban, DUP728, lefradafiban, sibrafiban, orbofiban, xemilofiban, lotrafiban (Class II) and an anticoagulant such as heparin selected from the group consisting of UFH or LMWH such as tinzaparin, certoparin, parnaparin, nadroparin, ardeparin, enoxaparin, reviparin, dalteparin, and fraxiparin or ultra LMWH, pentasaccharide, direct anti-Xa, direct anti-IIa (thrombin) or tissue factor pathway inhibitor (TFPI). Where in the anticoagulant is administered in reduced to sub-therapeutic amounts or amounts that provide a synergistic to additive improvement in the therapeutic index (efficacy/safety window).

Another object of the present invention is to provide a method of treating venous, arterial thrombosis in a mammal wherein the combination of (i) GPIIb/IIIa antagonists and (ii) anticoagulants listed above is administered in amounts to provide a synergistic effect on the efficacy and safety parameters.

Yet another object of the present invention is to provide a method of treating thrombosis in a mammal by administering a lyophilized formulation containing a GPIIb/IIIa antagonist compound and sub-therapeutic amounts of an anticoagulant for in-hospital (intravenous) and out-hospital (subcutaneous or oral).

### DETAILED DESCRIPTION OF THE INVENTION

The combinations of a lyophilized or liquid formulation of a GPIIb/IIIa antagonist with an anticoagulant such as UFH, low molecular weight heparin (LMWH), ultra LMWH, pentasaccharide, direct anti-Xa, direct anti-IIa (thrombin) or tissue factor pathway inhibitor (TFPI) at reduced amounts should have a tremendous impact in thrombosis. The anti-thrombotic benefits would include the prevention and treatment of venous and arterial thrombosis disorders including atherosclerotic arterial disease, valvular heart disease, cerebrovascular disease such as stroke, atrial fibrillation, coronary artery disease such as myocardial infarction and unstable angina, coronary artery bypass grafts, peripheral vascular disease, thromboembolic complications of prosthetic cardiovascular devices such heart valves and vascular grafts. These combinations are also expected to be useful in combining with endovascular stenting procedures such as percutaneous transluminal coronary angioplasty, to prevent subsequent arterial thrombus formation and re-occlusion, and to treat and prevent thrombotic complications in cancer patients. Also these lyophilized or liquid combinations should be useful in the treatment of thrombotic complications secondary catheters, surgery, and other drug-induced thrombosis.

UFH and Low molecular weight heparins such as tinzaparin or other LMWH derivatives useful in the combination of the present invention are commercially available and well known in the prior art. Specific examples of other useful GPIIb/IIIa antagonist compounds are eptifibatide, tirofiban, lefradafiban, sibrafiban, Orbofiban, lotrafiban, DUP728, DMP802, XV454, DMP754 (Roxifiban), XV459, and xemilofiban (Graul et la., Drugs of the Future 22: 508-517, 1997; Scarborough, Drugs of the Future 23: 585-590, 1998; Mousa and Wityak, Cardiovascular Drug reviews 16:48-61, 1998). Some of these examples are DUP728, DMP802, XV454, XV459, DMP754 are preferred. Others are readily apparent to those skilled in the art.

“Therapeutically effective amount” is intended to include an amount of a combination of compounds claimed effective to treat thrombosis in a mammal. The combination of compounds is preferably a synergistic combination. Synergy, as described for example by Chou and Talalay, Adv. Enzyme Regul. 22:27-55 (1984), occurs when the effect (in this case, an antithrombotic effect) of the compounds when administered in combination is greater than the additive effect of the compounds when administered alone as a single agent. In general, a synergistic effect is most clearly demonstrated at sub-therapeutic amounts of one or more of the combined compounds. Synergy can be in terms of anti-thrombotic effect, improved safety profiles or some other non-additive beneficial effect of the combination compared with the individual components in the same formulation.

By “administering in combination”, “combination”, or “combined” when referring to compounds described herein, it is meant that the compounds or components are administered together in the same formulation to the mammal being treated. By “sub-therapeutic amount,” it is meant that each component when administered to a mammal alone does not give the desired therapeutic effect for the disease being treated but when combined full therapeutic benefits are achieved.

### Dosage and Formulation

Combinations of GPIIb/IIIa antagonist (A) with anticoagulant (B), which might include the following: UFH or low molecular weight heparin (LMWH) or ultra LMWH, pentasaccharide or direct anti-Xa, anti-X/Xa or direct anti-IIa (thrombin) or tissue factor pathway inhibitor (TFPI) are administered for the prevention and treatment of thrombosis by any means that produces contact of the agents with their site of action.

Dosage forms of compositions suitable for administration contain from about 1 mg to about 100 mg of active ingredient per unit. In these pharmaceutical compositions the active ingredient (combination of GPIIb/IIIa antagonist, A and anticoagulant, B) will ordinarily be present in an amount of about 0.5-95% by weight based on the total weight of the composition. The active ingredient can be administered orally in solid dosage forms, such as capsules, tablets and powders, or in liquid dosage forms, such as elixirs, syrups and suspensions. It can also be administered parenterally, in sterile liquid dosage forms.

Gelatin capsules contain the active ingredient and powdered carriers, such as lactose. Starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance.

In general, water, suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts, and sodium EDTA. In addition, parenteral solutions can contain preservatives, such as benzalkonium chloride, methyl- or propyl-paraben and chlorobutanol. Suitable pharmaceutical carriers are described in Remington's Pharmaceutical Sciences, 17th ed., Mack Publishing Company, Easton, Pa., 1985, a standard reference text in this field, the contents of which are incorporated herein by reference.

Useful pharmaceutical dosage-forms for administration of the compounds of this invention can be illustrated as follows:

Capsules

A large number of unit capsules can be prepared by filling standard two-piece hard gelatin capsules each with 0.1 to 100 mg of active ingredient (GPIIb/IIIa antagonist, A and anticoagulant, B) 150 mg of lactose, 50 mg of cellulose, and 6 mg magnesium stearic. Additionally, other oral delivery enhancer might be added to improve the pharmacokinetics.

Soft Gelatin Capsules

A mixture of active ingredient (GPIIb/IIIa antagonist, A and anticoagulant, B) in a digestible oil such as soybean oil, cottonseed oil or olive oil can be prepared and injected by means of a positive displacement pump into gelatin to form soft gelatin capsules containing 0.1 to 100 mg of the active ingredient. The capsules should then be washed and dried. Additionally, other oral delivery enhancer might be added to improve the pharmacokinetics.

Tablets

A large number of tablets can be prepared by conventional procedures so that the dosage unit is 0.1 to 100 mg of active ingredient (GPIIb/IIIa antagonist, A and anticoagulant, B), 0.2 mg of colloidal silicon dioxide, 5 milligrams of magnesium stearate, 275 mg of microcrystalline cellulose, 11 mg of starch and 98.8 mg of lactose. Appropriate coatings may be applied to increase palatability or delay absorption. Additionally, other oral delivery enhancer might be added to improve the pharmacokinetics.

Suspension

An aqueous suspension can be prepared for oral administration so that each 5 mL contain 0.1 to 100 mg of finely divided active ingredient (GPIIb/IIIa antagonist, A and anticoagulant, B), 200 mg of sodium carboxymethyl cellulose, 5 mg of sodium benzoate, 1.0 g of sorbitol solution, U.S.P., and 0.025 mg of vanillin. Additionally, other oral delivery enhancer might be added to improve the pharmacokinetics.

Injectable

A parenteral composition suitable for administration by injection can be prepared by stirring 0.1 to 100 mg by weight of active ingredient (GPIIb/IIIa antagonist, A and anticoagulant, B) as lyophilized or soluble formulation. The solution is sterilized by commonly used techniques. The GPIIb/IIIa antagonist, A and the anticoagulant, B would be in the same vial or ampoule either in contact or separated by specific coating or by using a physical membrane barrier to be removed upon administration of the combination.

The formulation might include the following:

Citric acid, anhydrous sodium citrate, mannose, lactose, sodium hydroxide, acid, GPIIb/IIIa antagonist (A) and anticoagulant (B). The combined formulation might contain natural antioxidants.

The combined compounds (A and B) of this invention may be formulated such that, although the active ingredients are combined in a single dosage unit, the physical contact between the active ingredients is minimized. In order to minimize contact, for example, where the product is orally administered, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for combined compounds wherein one of the active ingredients is coated with a sustained-release material which affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of combined compounds in which the one compound is coated with a sustained and/or enteric release polymer, and the other compound is also coated with a polymer such as a low viscosity grade of hydroxypropyl methylcellulose or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric-coated compound and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the combined compounds, whether administered in a single dosage form or administered in separate forms but at the same time or concurrently by the same manner, will be readily apparent to those skilled in the art, based on the present disclosure.

Combination:

Each therapeutic compound (GPIIb/IIIa antagonist, A and anticoagulant, B) of this invention can be in any dosage form, such as those described above, and can also be administered in various ways, as described above. For example, the compounds may be formulated together (that is, combined together in one capsule, tablet, powder, or liquid, etc.) as a combination product.

Preferably, the route of administration of therapeutic combinations herein is intravenously, subcutaneously or orally.

As is appreciated by a medical practitioner skilled in the art, the dosage of the combination therapy of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above.

The proper dosage of a GPIIb/IIIa antagonist (A) and anticoagulant (B) including UFH, LMWH, ultra-LMWH, pentasaccharide, direct anti-Xa, anti-IIa, or TFPI combination is readily ascertainable by a medical practitioner skilled in the art, based upon the present disclosure. By way of general guidance, typically a daily dosage may be about 1 to 100 milligram of each component. By way of general guidance, when the compounds are administered in combination, the dosage amount of each component or the anticoagulant may be reduced. This reduced amount could be by about 20-80% relative to the usual dosage of the component when it is administered alone as a single agent for the treatment of thrombosis in view of the synergistic effect of the combination.

Obviously, numerous modifications and variations of the present invention are possible in light of the above teachings. It is therefore to be understood that within the scope of the appended claims, the invention may be practiced otherwise than as specifically described herein.

### EXAMPLES

### Example 1

The combination of unfractionated heparin (UFH) or LMWH ultra-LMWH, Pentasaccharide or modified heparin and a short acting GPIIb/IIIa antagonists such as Integrilin, tirofiban or DUP728 in the presence of polycationic carbohydrate such Chitosan, citric acid/sodium citrate, mannitol, and other non-active ingredients as described in the different dosage form section at pH 4-6 is a preferred formulation for intravenous administration.

### Example 2

The combination of UFH or LMWH, ultra-LMWH, Pentasaccharide or modified heparin and a long acting GPIIb/IIIa antagonists such as XV454, XV459 or other long acting GPIIb/IIIa antagonists in the presence of polycationic carbohydrate such Chitosan, citric acid/sodium citrate, mannitol and other non-active ingredients as described in the different dosage form section at pH 4-6 is a preferred formulation for intravenous and subcutaneous administration.

Results indicate that a combination of Compound (A) from the GPIIb/IIIa antagonist's classes and heparin or LMWH at their sub-therapeutic doses unexpectedly produced a significant antithrombotic effect (Mousa et al., Thrombosis & Haemostasis, 3441, 2001).

Additionally, a combination of both would improve the safety and reduced the thrombocytopenia that might be the result of heparin (HIT) or the result of a GPIIb/IIIa antagonist.

### Example 3

The combination of other anticoagulants including small molecule anti-Xa, anti-IX/IXa, anti-IIa or r-TFPI and a short acting GPIIb/IIIa antagonists such as Integrilin, tirofiban or DUP728 in the presence of citric acid/sodium citrate, mannitol, and other non-active ingredients as described in the different dosage form section at pH 4-6 is a preferred formulation for intravenous administration.

### Example 4

The combination of other anticoagulants including small molecule anti-Xa, anti-IX/IXa, anti-IIa or r-TFPI and a long acting GPIIb/IIIa antagonists such as XV454, XV459 or other long acting GPIIb/IIIa antagonists in the presence of polycationic carbohydrate such Chitosan, citric acid/sodium citrate, mannitol at pH 4-6 is a preferred formulation for intravenous, subcutaneous, oral, transdermal, intranasal or any other delivery mode of administration.

1. A method of treating thromboembolic disorders associated with arterial and venous thrombosis in a mammal comprising administering to said mammal a combination of: (A) of GPIIb/IIIa antagonist selected from the group consisting of eptifibatide, tirofiban, lefradafiban, sibrafiban, orbofiban, xemilofiban, DUP728, XV459, DMP754 (Roxifiban), DMP802, XV454 and (B) an anticoagulant.

2. A method for in hospital use of a short acting GPIIb/IIIa antagonists such as tirofiban, integrilin, DUP728 or any other short acting GPIIb/IIIa antagonist, with 2-4 hours half-life after termination of the IV bolus/infusion plus UFH or LMWH or any other short acting anticoagulant from the class of direct anti-Xa, direct anti-IX/IXa, direct anti-IIa or r-TFPI could be used as an IV injectable for the prevention and treatment of thromboembolic events associated with arterial (angina, MI, stroke, peripheral artery diseases) and venous thrombosis (DVT, PE) as a stand alone regimen or in conjunction with other therapies, post-surgery or with interventional procedures.

3. A method for In-hospital and out-hospital (intravenous, subcutaneous or oral) of a long acting GPIIb/IIIa antagonist (with >4 hours half life after the termination of IV bolus/infusion or SC injection) such as XV454, DMP802, XV459 or Roxifiban with LMWH such as tinzaparin, enoxaparin, dalteparin or any other long acting anticoagulant such as Pentasaccharide, direct anti-Xa, direct anti-IX/IXa, direct anti-IIa or r-TFPI could be used for the prevention and treatment of thromboembolic events associated with arterial and venous thrombosis.

4. The method of claims 1-3, wherein the combination of (A) and (B) provides an additive or synergistic effect.

5. The method of claims 1-3, wherein the combination administered is a combination of the GPIIb/IIIa antagonist and unfractionated heparin.

6. The method of claims 1-3, wherein the combination administered is a combination of the GPIIb/IIIa antagonist and a LMWH selected from the group consisting of tinzaparin, certoparin, parnaparin, nadroparin, ardeparin, enoxaparin, reviparin, reviparin, dalteparin, and fraxiparin.

7. The method of claims 1-3, wherein the combination administered is a combination of the GPIIb/IIIa antagonist and pentasaccharide.

8. The method of claims 1-3, wherein the combination administered is a combination of the GPIIb/IIIa antagonist and direct anti-Xa.

9. The method of claims 1-3, wherein the combination administered is a combination of the GPIIb/IIIa antagonist and direct anti-IIa.

10. The method of claims 1-3, wherein the combination administered is a combination of the GPIIb/IIIa antagonist and recombinant TFPI.

11. The method of claims 1-3, wherein the combination administered is a combination of the GPIIb/IIIa antagonist and ultra-LMWH or modified heparin or modified LMWH.

12. The method of claim 2, wherein the GPIIb/IIIa antagonist is either eptifibatide, tirofiban, lefradafiban, sibrafiban, orbofiban, xemilofiban, DUP728 or any other short acting GPIIb/IIIa antagonists.

13. The method of claim 3, wherein the long acting GPIIb/IIIa antagonist such as XV459, DMP754 (Roxifiban), DMP802, XV454 or any other long acting GPIIb/IIIa antagonist is administered in a sub-therapeutic amount.

14. The method of claims 1-3, wherein UFH is the standard generic heparin and the low molecular weight heparin is the generic tinzaparin, enoxaparin, daletparin or any other LMWH described earlier.

15. The method of claims 1-3, wherein the GPIIb/IIIa antagonist is either short acting such as DUP728, Tirofiban, Integrilin, DUP728 or Long acting such as XV459 (DMP754, roxifiban), DMP802, XV454 and as an anticoagulant, pentasaccharide.

16. The method of claims 1-3, wherein the GPIIb/IIIa antagonist is either short acting such as DUP728, Tirofiban, Integrilin, DUP728 or Long acting such as XV459 (DMP754, roxifiban), DMP802, XV454 and as an anticoagulant direct anti-Xa.

17. The method of claims 1-3, wherein the GPIIb/IIIa antagonist is either short acting such as DUP728, Tirofiban, Integrilin, DUP728 or Long acting such as XV459 (DMP754, roxifiban), DMP802, XV454 and as an anticoagulant, direct anti-IX/IXa.

18. The method of claims 1-3, wherein the GPIIb/IIIa antagonist is either short acting such as DUP728, Tirofiban, Integrilin, DUP728 or Long acting such as XV459 (DMP754, roxifiban), DMP802, XV454 and as an anticoagulant, direct anti-IIa.

19. The method of claims 1-3, wherein the GPIIb/IIIa antagonist is either short acting such as DUP728, Tirofiban, Integrilin, DUP728 or Long acting such as XV459 (DMP754, roxifiban), DMP802, XV454 and as an anticoagulant recombinant TFPI.

20. The method of claims 1-3, wherein the GPIIb/IIIa antagonist is either short acting such as DUP728, Tirofiban, Integrilin, DUP728 or Long acting such as XV459 (DMP754, roxifiban), DMP802, XV454 and as an anticoagulant, ultra-LMWH or modified heparin.

21. The use of a combination of: (i) a sub-therapeutic dose of GPIIb/IIIa antagonist selected from the group consisting of Abciximab, DMP754 (Roxifiban), XV454, XV459, DMP802 (i) and (ii) an anticoagulant selected from the claims 4-20.

22. The use of combinations listed in claims 4-20 in the prevention and treatment of venous and arterial thrombotic disorders as defined in detail in the invention.

23. The use of combinations listed in claims 4-20 in the prevention and treatment of thromboembolic events associated with arterial or venous thrombosis could be used as an injectable, oral, transdermal, inhalation, or by any of the routes listed in this invention.
